(19) United States
(12) Patent Application Publication
(10) Pub. No.: US 2003/0068297 A1
(43) Pub. Date: Apr. 10, 2003

(54) Composition and methods for skin rejuvenation and repair

(75) Inventor: Deepak Jain, San Diego, CA (US)

Correspondence Address: FOLEY & LARDNER, PO Box 80278, San Diego, CA, 92138-0278, US

(73) Assignee: DEEPAK JAIN, SAN DIEGO, CA (US)

(21) Appl. No.: 10/222,949

(22) Filed: Aug. 16, 2002

Related U.S. Application Data
(63) Continuation-in-part of application No. 60/313,306, filed on Aug. 18, 2001.
(63) Continuation-in-part of application No. 60/313,307, filed on Aug. 18, 2001.
(63) Continuation-in-part of application No. 60/313,313, filed on Aug. 18, 2001.
(63) Continuation-in-part of application No. 60/313,314, filed on Aug. 18, 2001.

(51) Int. Cl. A61K 38/19; A61K 38/18; A61K 31/557; A61K 31/728; A61K 31/715

(52) U.S. Cl. 424/85.1; 514/12; 514/54; 514/573

(57) ABSTRACT

The present invention provides compositions for the repair of mammalian skin. The compositions contain cell growth enhancers to increase the growth rate of skin cells, nutrients to support log phase growth of skin cells, extracellular matrix proteins, stimulators of extracellular matrix proteins, and penetration enhancers. The compositions of the present invention are effective for repairing and rejuvenating mammalian skin, such that aging skin treated with the compositions has a significant reduction in the number of fine lines and wrinkles in the skin. The compositions are also effective for promoting the healing of skin that has suffered a wound, such as a sunburn or abrasion, and for promoting the growth of hair on the scalp.

This application is a continuation-in-part of U.S. Application Serial No. 60/313,306 filed Aug. 18, 2001, and a continuation-in-part of U.S. Application Serial No. 60/313,307, filed Aug. 18, 2001, and a continuation-in-part of U.S. Application Serial No. 60/313,313, filed Aug. 18, 2001 and a continuation-in-part of U.S. Application Serial No. 60/313,314, filed Aug. 18, 2001, each of which is hereby incorporated by reference in their entireties.

### FIELD OF THE INVENTION

The present invention relates to the fields of personal and topical skin care, cosmetics, cosmeceuticals, skin rejuvenation, skin anti-aging, skin repair, and wound healing.

### BACKGROUND OF THE INVENTION

The following discussion of the background of the invention is merely provided to aid the reader in understanding the invention and is not admitted to describe or constitute prior art to the present invention.

Skin care products in the market claim to rejuvenate, regenerate and repair skin through various additives included in cosmetic and cosmeceutical formulations but generally camouflage the signs of aging skin. Some products claim to rejuvenate skin cells with cosmeceutical additives such as vitamins, hydroxy acids, and botanical extracts. Many of these products make broad claims, usually without scientific basis. Vitamins and hydroxy acids primarily act as exfoliants and facilitate the shedding of the surface cells to produce younger looking skin (U.S. Pat. No. 5,547,988). But these products provide only a temporary solution and only produce a superficial effect. Botanical extracts and herbal components are popular additives claiming biological skin rejuvenation but no active components are defined in these products and results are varied at best (U.S. Pat. No. 6,036,966). Other products claim to regenerate skin with biological ingredients such as growth factors. But growth factors are large protein molecules and are unable to penetrate the epidermis of the skin. Furthermore, these products lack specific delivery systems for delivering the growth factors to reach the target skin cells and therefore do not penetrate the skin. Consequently, they fail to demonstrate any beneficial effects.

A more fundamental and comprehensive approach is needed for treating aging skin that is based on science and the biology of the skin. Skin aging is a natural phenomenon that occurs over time. The primary element responsible for accelerating skin aging is overexposure to the sun's harmful rays causing photo damage. Photoaging can be slowed with the use of sunscreens and avoidance of sun exposure. But the more important and complex causes of skin aging are biological and are caused by a slowing of the division rate of skin cells and defective cross-linking of collagen and elastin fibers in the skin. With age, the skin fails to regenerate itself as quickly as it used to, and shows common signs of aging in terms of tone and texture. Also, collagen and elastin fibers in the underlying layers of the skin, which provide the scaffolding for the surface layers, begin to weaken and deteriorate with age causing the skin to lose elasticity and form sags, fine lines, and wrinkles. Thus, from a biological standpoint, an effective plan for rejuvenating and repairing skin must address the rejuvenation of skin cells at both the epidermal and the dermal layers, stimulation of the production of skin matrix elements, and the sustainability of the rejuvenated conditions over the long term. Biologically based components such as large molecular weight proteins are unable to cross the skin barrier naturally. Comprehensive skin care, therefore, must also address penetration of the components through the skin permeability barrier.

### SUMMARY OF THE INVENTION

The present invention provides compositions for the repair of mammalian skin. The compositions contain cell growth enhancers to increase the growth rate of skin cells, nutrients to support log phase growth of skin cells, extracellular matrix proteins, stimulators of extracellular matrix proteins, and penetration enhancers. The compositions of the present invention are effective for repairing and rejuvenating mammalian skin, such that aging skin treated with the compositions has a significant reduction in the number of fine lines and wrinkles in the skin. The compositions are also effective for promoting the healing of skin that has suffered a wound, such as a sunburn, cut, scrape, or abrasion.

In a first aspect, the present invention provides compositions for skin repair. The compositions contain an amount of at least one cell growth enhancers effective to increase the growth rate of skin cells. In various embodiments the cell growth enhancers are one or more of epidermal growth factor (EGF), fibroblast growth factor-α (FGF-α), insulin-like growth factor (IGF), granulocyte colony stimulating factor (GCSF), granulocyte macrophage colony stimulating factor (GMCSF), platelet derived growth factor (PDGF), keratinocyte growth factor (KGF), fibroblast growth factor (FGF) (acidic and basic), tissue growth factor-α (TGF-α), vascular endothelial growth factor (VEGF), other growth factors, growth hormone, erythropoietin, hematopoietin, prostaglandin, cytokines, hyaluronic acid, fibronectin, vitronectin, laminin, tenasin, regulatory factors, angiogenic factors, and adhesion proteins. The compositions also contain at least one nutrient effective to support log phase growth of skin cells. In various embodiments the nutrients can be one or more of carbohydrates, lipids, essential and non essential amino acids, salts, vitamins, minerals, trace metals, nucleosides, purines, pyrimidines, glutathione, peptides, peptones, lipoproteins, fatty acids, serum substitutes, or analogs and derivatives thereof. The compositions also contain at least one extra-cellular matrix protein that, in various embodiments, are one or more of fibrous proteins, collagen, elastin, adhesion proteins, glucosamineglycans, proteoglycans, and integrins. The compositions further contain at least one stimulator of extra-cellular matrix effective to increase the production of extra-cellular matrix in the skin. In various embodiments, these stimulators are one or more of tissue growth factor-β, and adhesion proteins. The compositions further contain penetration enhancers effective to allow the penetration of the cell growth enhancers, nutrients, extracellular matrix proteins, and stimulators of extracellular matrix into and through the epidermal layer of mammalian skin in amounts effective to promote and achieve skin repair. In various embodiments, the stimulators are one or more of mineral oil, fatty alcohols, lipids, fatty acids, oils, detergents, alcohols, lipoic acids, glycols, a transdermal delivery vehicle, or a transdermal delivery device. The compositions are provided in a biologically acceptable carrier for topical application.

The term “cell growth enhancers” used herein means components that increase the rate of growth of the cells. Rate of growth is determined by the amount of time necessary for a population of cells to double in number. Normal doubling times for young, healthy fibroblast cells are 24-30 hours. An amount of cell growth enhancers “effective to increase the growth rate of skin cells” is an amount effective to lower the doubling time of a population of fibroblasts that are doubling one time in more than every 35 hours from more than 35 hours to less than 32 hours. In another embodiment, the amount effective to increase the growth rate will decrease the doubling time of a population of fibroblasts by at least 10% or by at least 15% or by at least 20%. Cell growth enhancers include, but are not limited to, growth factors (e.g., epidermal growth factor (EGF), keratinocyte growth factor (KGF), fibroblast growth factor (acidic and basic) (FGF), insulin-like growth factor (IGF), platelet derived growth factor (PDGF), granulocyte colony-stimulating factor (G-CSF), granulocyte-macrophage colony-stimulating factor (GM-CSF)), cytokines such as hepatopoietin and erythropoietin, regulatory factors such as human growth hormone and prostaglandin, angiogenic factors such as vascular endothelial growth factor (VEGF), and adhesion proteins such as fibronectin, vitronectin, laminin, and tenasin. In various embodiments the compositions contain these constituents in their native forms, but can also contain stimulators of these constituents. For example, amphiregulin and/or tissue growth factor alpha (TGF-α) may be used to induce and stimulate EGF production in cells.

An amount of one or more nutrients is provided in the compositions effective to support log phase growth of human fibroblasts or keratinocytes. Log phase growth is characterized by an exponential multiplication in the number of cells. “Nutrients” include, but are not limited to, one or more of the following: monosaccharides, disaccharides, polysaccharides, essential and non-essential amino acids (D and/or L) and their derivatives, proteins and protein extracts, lipids and lipoproteins, fatty acids, vitamins, minerals and trace metals such as ferrous and ferric compounds (e.g., nitrates and sulfates), complex sources of carbon and nitrogen such as peptones and egg yolk, deoxyribose, ribose, nucleosides, riboflavin, insulin, transferrin, acetate salts, phosphate salts, pyruvate salts, buffers, cholesterol, 2-mercaptoethanol, pyrimidines, purines, glutathione. Nutrients can also include serum substitutes (e.g., bovine serum albumin), natural extracts, plant and animal derived components. In some embodiments nutrients include the components above in a modified form, for example modified to give hydrophilic, hydrophobic and/or lipophilic properties to enhance penetration into the skin. For example, glucose can be modified to octyl glucose, which enhances its delivery to the inner layers of the skin. Likewise, other modified components such as cetyl ascorbate or cetyl phosphoryl ascorbate can be used instead of (or in addition to) ascorbate. Also, lipophilic analogs of amino acids may be used.

The term “extracellular matrix (ECM) proteins,” as used herein means proteins that provide the structure to the skin. Extracellular matrix proteins provide the cellular support structure or “scaffolding” underneath the epidermal layer of skin cells, and their degradation plays an important role in wrinkle formation of aging skin. ECM proteins include, but are not limited to, fibrous proteins, adhesion proteins, glucosamineglycans and proteoglycans, and integrins. “Fibrous proteins” are insoluble proteins, including the collagens, elastins, and keratins that are involved in structural or fibrous tissues. “Adhesion proteins” are a large family of proteins that mediate direct contact between cells or between cells and the extracellular matrix during such physiological processes as cell activation, migration, proliferation, and differentiation. Examples include, but are not limited to, fibronectin, vitronectin, thrombospondin, laminin, and tenasin. Compositions of the invention use components in native forms or analogs thereof.

The term “stimulators of extracellular matrix protein production” as used herein means components that increase the production of extracellular matrix proteins by fibroblasts or keratinocytes. Stimulators of ECM protein production include, but are not limited to, growth factors, adhesion proteins and isoforms, peptides and derivatives thereof. Growth factors include transforming growth factor-β (TGF-β) and isoforms like β1, β2, β3, and vascular endothelial growth factor (VEGF). A stimulator is “effective to increase the production of extracellular matrix proteins” when it increases the production of extracellular matrix proteins in fibroblasts or keratinocytes by 10% or more.

The term “penetration enhancers” as used herein means a compound that facilitates the movement of substances into and/or through the epidermis of the skin. Examples of penetration enhancers include, but are not limited to, lipids, lipoproteins, fatty acids and fatty alcohols, detergents, alcohols, glycols, mineral oils, essential oils, a transdermal delivery vehicle or device. Lipids include, but are not limited to, linoleic acid, oleic acid, and chea butter. Alcohols include, but are not limited to, methanol, ethanol, propanol, isopropanol, glycols and analogs and derivatives thereof. Detergents include, but are not limited to, Tween 80, Triton-X100, sodium dodecylsulfate (SDS), and sulfated higher alcohols or derivatives thereof. Glycols include, but are not limited to, short chain glycols such as propylene glycol and butylene glycol. Oils include, but are not limited to, such as herbal, animal, synthetic, natural, essential, and mineral oils. Components may be linked to a TAT protein or part thereof, or a peptide, etc. to improve transport through the skin. Likewise, VEGF may be linked to a detergent molecule such as Tween 80 for enhanced delivery. The term “penetration enhancers” also includes those compounds described above chemically modified to impart hydrophilic, hydrophobic and/or lipophilic properties to the compounds. The term also includes derivative, analogs, isoforms, precursors and subunits of the compounds described above.

The term “biologically acceptable carrier” as used herein, means a carrier suitable for topical application to mammalian skin. Thus, a biologically acceptable carrier can be applied to mammalian skin without causing undue toxicity, irritation, allergic response, and the like. The formulations of the invention may be prepared in combination with additional ingredients such as sunscreens, moisturizers, exfoliators, cosmeceuticals, and the addition of appropriate carriers and bulking agents such as gums, resins, waxes, polymers, salts, and the like. Formulations may be composed in the form of sprays using either mechanical pump containers or pressurized aerosol containers using conventional propellants.

The term “skin cells” as used herein means keratinocytes and fibroblasts. “Corneocytes” are the dead keratin-filled squamous cells of the stratum comeum. The term “skin rejuvenation” or “rejuvenation of skin” refers to the prevention or reduction of fine lines and wrinkles in the skin, and that fine lines and wrinkles are reduced by 10% or more in number. The number of fine lines and wrinkles is calculated according to methods known in the art, such as using D-SQUAME® tape and image analysis (e.g., as in Example 11). “Fine lines” are the lines that appear in mammalian skin due to the effects of aging. Wrinkles are deeper than fine lines. The term “skin repair” refers to skin rejuvenation, the healing of a wound, or the moisturization of skin. The wound may be, for example, caused by sunburns, cuts, bruises, scrapes, chemical bums from cosmetic facial peels or other dermatological and cosmetic surgery procedures. The term “wound healing” or “healing of a wound” as used herein refers to the repair of wounds due to a break in the skin barrier or due to a bum, for example, by cuts, sunburns, ulcer wounds, or wounds received during a surgical procedure. The term “topical application”, as used herein, means any means of application to the surface of the skin. “Moisturization” refers to a 5% or greater increase in the moisture level of the skin, as measured with a NOVA DPM (dermal phase meter) meter or equivalent (Nova Technologies). In other embodiments, moisturization refers to a 10% or greater or 15% or greater or 20% or greater increase in the moisture level of the skin, using the same measurement. “Angiogenic factors” are factors that stimulate the formation of new blood vessels. “Mineral oil” is any oil made from mineral sources, e.g., petroleum. A “fatty alcohol” is an alcohol with long, unbranched carbon chains, derived mainly from petrochemical products. “Essential oils” are plant products, usually somewhat volatile, giving the odors and tastes characteristic of the particular plant, thus possessing the essence, e.g., citral, pinene, almond oil, coconut oil, linseed oil, camphor, menthane, terpenes, usually, the steam distillate or plants or oils of plants obtained by pressing out the rinds of a particular plant. A “proteoglycan” is a complex molecule containing at least one glucosaminoglycan bound to a core protein. Proteoglycans include, but are not limited to, versican, decorin, betaglycan, syndecan and aggrecan. “Glucosaminoglycans” are heteropolysaccharides which contain an N-acetylated hexosamine in a characteristic repeating disaccharide unit. The repeating structure of each disaccharide involves alternate 1,4- and 1,3-linkages consisting of either N-acetylglucosamine or N-acetylgalactosamine. Glucosamineglycans include, but are not limited to, chondroitin sulfate, dermatan sulfate, heparin sulfate, heparin, keratin sulfate and hyaluronan. “Lipoproteins” are particles composed of proteins and lipids (triglycerides, phospholipids and cholesterol) that enable lipids (which are water insoluble) to be carried in blood plasma. “Minerals” are naturally occurring chemical compounds or a limited mixture of chemical compounds that form crystals and have specific physical and chemical properties that can be used to identify them. “Peptones” are any of the various products produced as a result of partial hydrolysis of proteins. “VEGF” is a protein that stimulates the growth of new blood vessels. “EGF” is a protein that stimulates epidermal cells to divide. “FGF” is a growth factor that has been isolated from a variety of cells. It has potent heparin-binding activity and is a potent inducer of DNA synthesis in a variety of normal diploid mammalian cell types from mesoderm and neuroectoderm lineages. It also has chemotactic and mitogenic activities. FGF has acidic and basic forms. “IGF” refers to insulin-like growth factors I and II. Insulin like growth factors I and II are polypeptides with sequence similarity to insulin. They are capable of eliciting the similar biological responses, including mitogenesis in cell culture. “GCSF” is a glycoprotein containing internal disulfide bonds. It induces the survival, proliferation, and differentiation of neutrophilic granulocyte precursor cells and functionally activates mature blood neutrophils. “GMCSF” is an acidic glycoprotein with internal disulfide bonds. It stimulates the production of neutrophilic granulocytes, macrophages, and mixed granulocyte macrophage colonies from bone marrow cells and can stimulate the formation of eosinophil colonies from fetal liver progenitor cells. It also has some functional activities in mature granulocytes and macrophages. “PDGF” is an important mitogen that promotes growth in culture of cells of connective tissue origin. It consists of 2 different but homologous polypeptides A and B (˜30,000 D) linked by disulfide bonds, and plays a role in wound healing. “KGF” is a growth factor structurally related to fibroblast growth factor. “Cytokines” are non-antibody proteins that act as intercellular mediators. They differ from classical hormones in that they are produced by a number of tissue cell types rather than by specialized glands. They generally act locally in a paracrine or autocrine rather than endocrine manner. “Regulatory factors” are proteins active in the activation or repression of transcription of the gene. “Angiogenic factors” are proteins that act to vascularize tissue and act in the development of new capillary blood vessels. “Carbohydrates” are the class of aldehyde or ketone derivatives of polyhydric alcohols, usually having hydrogen and oxygen in the proportion to form water, Cn(H₂O)n. “Amino acids” are organic compounds that generally contain an amino (—NH2) and a carboxyl (—COOH) group and are the subunites that polymerize to form proteins. “Salts” are the neutral compounds formed by the union of an acid base. “Vitamins” are essential organic compounds required in trace amounts for normal growth and metabolic processes. They usually serve as components of coenzyme systems. “Purines” are a series of heterocyclic compounds that are variously substituted in nature and and are known also as purine bases. They include adenine and guanine. “Pyrimidines” are a family of 6-membered heterocyclic compounds. They are planar and aromatic in character and include several nucleic acid constituents (cytosine, thymine, and uracil). “Glutathione” is the tripeptide glutamylcysteinylglycine. “Peptides” are any member of a class of compounds which yield two or more amino acids on hydrolysis. They are formed by loss of water from the NH2 and COOH groups of adjacent amino acids. Peptides form the constituent parts of proteins. “Fatty acids” are organic, monobasic acids derived from hydrocarbons by the equivalent of oxidation of a methyl group to an alcohol, aldehyde, and then acid. Fatty acids are saturated and unsaturated. “Collagen” is the protein substance of the white fibres (collagenous fibres) of skin, tendon, bone, cartilage, and all other connective tissue, composed of molecules of tropocollagen, it is converted into gelatin by boiling. “Elastin” is a glycoprotein that is randomly coiled and cross-linked to form elastic fibres that are found in connective tissue. Like collagen, elastin composition is high in proline content. “Integrins” are a superfamily of cell surface proteins. Most integrins are heterodimeric with a subunit of about 95 kD that is conserved through the superfamily and a more variable subunite of 150-170 kD. “Detergents” are agents that are usually salts of long chain aliphatic bases or acids, that exert a cleansing (oil-dissolving) and antimicrobial effects through a surface action that depends on possessing both hydrophilic and hydrophobic properties. “Alcohols” are alkyl compounds containing a hydroxyl group.

Many embodiments of the present compositions are possible. In various embodiments, the cell growth enhancers are one or more of epidermal growth factor (EGF), fibroblast growth factor-α, (FGF-α), insulin growth factor (IGF), hyaluronic acid, fibronectin, alcohol, granulocyte colony stimulating factor (GCSF), granulocyte-macrophage colony stimulating factor (GMCSF), (platelet derived growth factor) PDGF, keratinocyte growth factor (KGF), fibroblast growth factor (FGF), tissue growth factor-α (TGF-α), and isoforms, vascular endothelial growth factor (VEGF), ascorbate, and derivatives thereof. The cytokines are hepatopoietin and/or erythropoietin, the regulatory factors are growth hormone or a prostaglandin, the angiogenic factor is VEGF; and the adhesion proteins are fibronectin, vitronectin, laminin and/or tenasin.

In various embodiments the nutrients are one or more of D-glucose, amino acids, sodium chloride, potassium chloride, sodium pyruvate, vitamin B12, choline chloride, inositol, calcium chloride, magnesium sulfate, ferric nitrate, ferrous sulfate, zinc sulfate, cupric sulfate, hypoxanthine, linoleic acid, lipoic acid, inositol, oleic acid, collagen, insulin, and transferrin. In various embodiments, the extracellular matrix proteins are one or more of collagen, elastin, and transferrin, the stimulator of extracellular matrix protein production is ascorbate, and the penetration enhancers are one or more of propylene glycol, a fatty alcohol, polyoxyethylene sorbitan monooleate (Tween 80™), butylene glycol, mineral oil, and essential oils. The penetration enhancer can also be a portion of a TAT protein sequence attached to a component protein.

In another aspect the present invention provides methods for repairing mammalian skin. The methods include contacting the skin with a composition of the invention, allowing the composition to remain in contact with the skin for a period of time sufficient for the cell growth enhancers, nutrients, extracellular matrix proteins, and stimulators of extracellular matrix protein production to permeate mammalian skin in amounts effective to repair the skin, and thereby repair the mammalian skin. In preferred embodiments of the invention, the mammal is a human. In other embodiments, the compositions are applied as a coating on medical or surgical devices, such as sutures, implants, homeostatic plugs, dressings, gauze and pads. A “biologically effective amount” is an amount effective to perform the function that is described for the individual component. In various embodiments the compositions can be applied through repeated applications, such as each evening at bedtime, or daily. The compositions can remain on the skin for a convenient period of time, for example, 6 hours, 8 hours, 10 hours, 12 hours, or any period of time the user deems convenient.

In another aspect the present invention provides methods for increasing hair growth on the scalp. The methods include contacting the skin of the scalp with a composition of the invention, allowing the composition to remain in contact with the skin for a period of time sufficient for the cell growth enhancers, nutrients, extracellular matrix proteins, and stimulators of extracellular matrix to permeate mammalian skin in amounts effective to increase hair growth, thereby increasing the growth of hair on the scalp. An increase in the growth of hair on the scalp means a 5% or greater increase in weight of hair clippings or hair count on the treated scalp area. In various embodiments, the clippings can be taken after 3 months or 6 months of treatment, or any period of time sufficient to provide a statistically meaningful result. In preferred embodiments, persons with healthy hair on the scalp can realize an increase in hair growth. The present methods are also useful for preventing hair loss. “Preventing hair loss” means that the loss of hair from the scalp is slowed.

The summary of the invention described above is not limiting and other features and advantages of the invention will be apparent from the following detailed description of the preferred embodiments, as well as from the claims.

### DETAILED DESCRIPTION OF THE INVENTION

The present invention relates to the field of topical skin care, cosmetics, cosmeccuticals, skin rejuvenation, hair care, skin anti-aging, skin repair, and wound healing. The invention directly addresses the more important and complex causes of skin aging caused by the age-induced slowing of the division rate of the skin cells and the defective cross-linking of collagen and elastin fibers. The compositions described herein provide skin care compositions that rejuvenate skin cells at both the epidermal and the dermal layers, stimulate skin cells to increase extracellular skin matrix production, and enhance the penetration of composition ingredients through the skin permeability barrier.

The compositions of the present invention are formulated for comprehensive skin rejuvenation and skin repair, and contain a unique combination of components provided in a biologically acceptable carrier. In preferred embodiments the compositions are topically applied to the surface of treated skin. The composition ingredients form the building blocks for complete care of skin cells from a scientific and biological standpoint.

Cell growth enhancers provided in the compositions increase the growth rate of skin cells to approach the rate of growth observed in the skin of persons below 30 years of age. Nutrients and nutritional factors provided in the composition provide nourishment and energy to maintain cells in the rejuvenated mode. Extracellular matrix proteins and stimulators of extracellular matrix protein production serve to replenish, produce and maintain matrix and structure of the skin. Penetration enhancers ensure that components penetrate the skin barrier and are delivered to the skin cells. The compositions may also contain components extracted from prokaryotic and eucaryotic cultures, such as proteins, lipoproteins or glycosylated protein fractions. The compositions are formulated to effective concentrations so as to provide rejuvenating effects on the skin such that normal, youthful, skin function is achieved and maintained. Since these compositions contain the essential building blocks for skin repair, they find wide applications in skin repair as in treating sunburns, radiation-bums, scrapes, superficial bums, or for use in cosmetic surgery procedures such as facial peels. The compositions also find application in wound healing such as repair of cuts, bums, ulcer and incision wounds, and can also be applied as a coating on medical or surgical instruments such as threads for sutures, prosthetic implants, homeostatic plugs, and wound dressings.

In a highly preferred embodiments the compositions may be used to prevent or reverse the physical effects of skin aging. Examples include, but are not limited to, topical applications of the compositions to maintain youthful skin texture and prevent fine lines and wrinkles from forming as a person ages. In a preferred embodiment the compositions may be used in the rejuvenation of aged skin. For example, the compositions can be topically applied to improve skin texture and reduce the number of fine lines and wrinkles. In another embodiment the compositions can contain varying concentrations of the constituents in the treatment of different skin conditions. For example, one embodiment can contain a higher concentration of enhancers and stimulators to increase cell growth. In another embodiment the composition can contain a higher concentration of nutrients for maintaining reactivated cells. In yet another embodiment the compositions can contain a higher concentration of extracellular matrix and stimulators of extracellular matrix protein production to reduce the number of lines in heavily wrinkled skin. A formulation containing balanced concentrations in the composition may be used to maintain rejuvenated, youthful skin.

In another embodiment the compositions may be used for the care of skin of the scalp that bears hair. For example, the compositions are topically applied to the scalp to prevent hair loss or increase hair growth from the follicles.

In another embodiment the compositions may be applied for the repair of damaged skin. Damaged skin is skin that has suffered a trauma, such as an abrasion, sunburns, scrapes, superficial burns. The compositions can also be used to aid the healing of skin after cosmetic and reconstructive surgery procedures, or after a cosmetic facial peel procedure. Other examples of healing damaged skin include, but are not limited to applications to promote healing of wounds such as cuts, bums, ulcer and incision wounds. The compositions of the invention are topically applied to the skin to be treated. The compositions can be administered as a film, mask, spray, or ointment. In another embodiment the compositions are used as a coating of medical or surgical devices, such as sutures, implants, homeostatic plugs, dressings, gauze and pads.

Stimulators of adhesion proteins comprise hyaluronic acids, fibronectin, vitronectin, and versican. Compositions use components in native forms or substitutes, but preferably use inducers, activators, and precursors of these components. For example ascorbate or an analog of ascorbate may be used to induce TGF-β, analogs of Retin-A may be used to deposit collagen. In another example, a stress protein such as GR78, or a peptide thereof, may be used to induce TGF-β. Hyaluronic acid and fibronectin may be used to stimulate glucosamineglycans and proteoglycans.

The compositions may also be formulated in oils as emulsions for improved penetration and delivery. The term “delivery vehicle” as used herein means a mechanism for delivering the compositions to the skin cells. Examples of delivery vehicles include, but are not limited to, liposomes, micelles, emulsions and micro-emulsions containing one or more of the composition ingredients. For example, emulsion carriers including, but not limited to, oil-in-water, water-in-oil, water-in-oil-in-water, and oil-in-water-in-silicone emulsions are useful in the present invention. Other examples of delivery vehicles include, but are not limited to aqueous solutions such as hydro-alcoholic solvent systems and non-aqueous solutions such as oil, ethanol, isopropanol, dimethicone, cyclomethicone, dimethlysulfoxide, solids, powders, gels or films, serum, cream, or silicones. The term “delivery device” as used herein means devices that are used to deliver the compositions to the skin cells. Examples of delivery devices include, but are not limited to, electrical devices such as ionotophoresis, pumps, a transdermal patch, an occlusive pad, mask or dressing, adhesive or non-adhesive bandage, a gel or film.

Preparations and Applications

The compositions of the present invention are prepared by mixing of the ingredients in one or more steps, with or without heating or cooling. Moisturizers, absorption enhancers, sunscreens, color, preservatives, and fragrance may also be added to the formulations as desired.

The compositions of the invention are useful for rejuvenating skin cells to prevent, and/or slow down skin aging effects, improve skin texture and prevent and reduce fine lines and wrinkles, and rejuvenate hair follicles and prevent hair loss. The compositions are for general cosmetic use and for specific skin treatments. For example a treatment regimen may use formulations of nutrients, cell growth enhancers, and stimulators of extra-cellular matrix production to rejuvenate aging skin. A composition rich in nutrients may be used for a defined period to energize the cells and prepare them for application of the complete composition. A composition especially rich in cell growth enhancers may be used to improve cell growth in aging skin. A composition rich in extracellular matrix stimulators may be used to improve skin structure. In preferred embodiments, formulations containing a balanced mixture of the ingredients mentioned above may be used to achieve and maintain healthy, rejuvenated skin. The compositions of the invention are also useful for the prevention of hair loss from the skin of the scalp, or to decrease the rate of hair loss from the scalp.

The compositions of the invention are useful for repairing damaged skin. For example, topical applications may be used to promote healing of damaged skin. Other topical applications include healing of facial peels and other cosmetics surgery procedures, healing of non-diseased wounds such as cuts, bums, ulcer and incision wounds, or as a wound healing promoter such as a coating of medical or surgical device essentially consisting of sutures, implants, homeostatic plugs, dressings, gauze and pads. In any or all of these applications the composition may be administered as a gel, film or mask, spray or ointment.

The invention is further illustrated in the following examples. These examples are given solely for the purpose of illustration and are not to be construed as limitations of the present invention as many variations thereof are possible without departing from the spirit and scope of the invention.

The following are exemplary compositions in accordance with this invention. Many variations of compositions, components and ingredients (selected from groups stated in description of terms) can be effectively employed for the applications of the invention. Amounts are given as examples only and higher and lower amounts can also be effectively employed. Formulations may contain commonly used cosmetic ingredients such as sunscreens, moisturizers, exfoliators, active cosmeceuticals, preservatives, antibacterial agents, anti-fungal agents, antiviral agents, antibiotics, color, fragrance and appropriate carriers and bulking agents such as gums, resins, waxes, polymers, salts, and the like.

### EXAMPLE 1

This example provides an example of a composition for rejuvenating skin. This composition rejuvenates the skin and improves the texture of the skin. The composition is prepared as an emulsion with preservatives, as illustrated below. The person of ordinary skill will realize that many variations of this preparation can be made by varying the ingredients to address particular needs of a situation.

| COMPONENTS | INGREDIENT | Conc. Range (mg/L) | |
|---|---|---|---|
| Nutrients | | | |
| Sugar | D-Glucose | 2.0-6.0 | g/L |
| Amino acids | essential and non essential | 4.0-150.0 | |
| Salts | Sodium chloride, Potassium | 300.0-5000.0 | |
| | chloride | | |
| Pyruvate salt | Sodium Pyruvate | 50.0-60.0 | |
| Phosphate salts | Sodium phosphate | 50.0-80.0 | |
| Vitamins | Selected, B12, Choline chloride, | 0.5-15.0 | |
| | Inositol | | |
| Minerals | Calcium chloride, Magnesium | 25.0-150.0 | |
| | sulfate | | |
| Trace metals | Ferric nitrate, Ferrous, Zinc and | 0.001-0.6 | |
| | Cupric sulfate | | |
| Nucleosides | selected | 0.001-0.01 | |
| Purines | Hypoxanthine | 2.0-4.0 | |
| Fatty acids | Linoleic Acid, Lipoic acid | 0.03-0.3 | |
| Proteins | Collagens, Insulin, Transferrin | 0.1-3.0 | |
| Cell growth | | | |
| enhancers | | | |
| Growth factor | EGF, FGF | 0.1-10.0 | |
| Attachment factor | Hyaluronic acid | 1.0-20.0 | |
| ECM and | | | |
| Stimulators | | | |
| Growth factor | TGF-β | 0.1-10.0 | |
| Fibrous Protein | Collagen | 0.1-3.0% | |
| ECM stimulator | Ascorbate | 30-150 | |
| Adhesion protein | Fibronectin | 5.0-50.0 | |
| Glucosamine | Heparin | 0.1-10 | |
| glycan | | | |
| Proteoglycan | Aggrecan | 0.1-10 | |
| Penetration | | | |
| Enhancers | | | |
| Propylene glycol | | 0.1-4.0 | |
| Fatty Alcohol | | 0-20.0 | |
| Tween 80 | | 0-5.0 | |
| Essential oil | | 10-90% | |
| (oil-in-water | | | |
| emulsion) | | | |
| Other Ingredients | | | |
| Extracts | Xanthan gums | 0.1-1.0 | |

| | |
|---|---|
| Water (USP) | Balance |

### EXAMPLE 2

This example provides an example of a composition suited for skin repair, such as the healing of superficial burns caused by facial peels. The formulation is prepared as a cream with preservatives, as illustrated below.

| COMPONENTS | INGREDIENT | Conc. Range (mg/L) | |
|---|---|---|---|
| Nutrients | | | |
| Sugar | D-Glucose | 2.0-6.0 | g/L |
| Amino acids | essential and non essential | 4.0-150.0 | |
| Vitamins | B12, Choline chloride, Inositol | 0.5-15.0 | |
| Buffer | Sodium bicarbonate | 2.0-3.0 | g/L |
| Minerals | Calcium chloride, Magnesium | 25.0-150.0 | |
| | sulfate | | |
| Trace metals | Ferric nitrate, Ferrous, Zinc and | 0.001-0.6 | |
| | Cupric sulfate | | |
| Nucleosides | selected | 0.001-0.01 | |
| Purines | Hypoxanthine | 2.0-4.0 | |
| Fatty acids | Oleic acid | 0.03-0.3 | |
| Proteins | Collagens, Insulin, Transferrin | 0.1-3.0 | |
| Cell growth | | | |
| enhancers | | | |
| Growth factor | TGF-α, EGF | 0.1-10.0 | |
| Attachment factor | Hyaluronic acid | 1.0-20.0 | |
| ECM and | | | |
| Stimulators | | | |
| Growth factor | TGF-β, VEGF | 0.1-10.0 | |
| Fibrous Protein | Collagen | 0.1-3.0% | |
| ECM stimulator | Ascorbate | 30-150 | |
| Adhesion protein | Hyaluronan | 5.0-50.0 | |
| Glucosamine | Heparin | 0.1-10 | |
| glycan | | | |
| Proteoglycan | Aggrecan, heparin | 0.1-10 | |
| Penetration | | | |
| Enhancers | | | |
| Butylene glycol | | 0.1-4.0 | |
| Tween 80 | | 0-5.0 | |
| Synthetic oil | | 10-90% | |
| Others | | | |
| Extracts | Xanthan gums | 0.1-1.0 | |

| | |
|---|---|
| Water (USP) | Balance |

### EXAMPLE 3

This example provides a composition for wound healing, such as cuts, surgical incisions, or other breaks in the skin. The formulation is prepared as an ointment with an antimicrobial agent or antibiotics.

| COMPONENTS | INGREDIENT | Conc. Range (mg/L) | |
|---|---|---|---|
| Nutrients | | | |
| Sugar | D-Glucose | 2.0-6.0 | g/L |
| Amino acids | essential and non essential | 4.0-150.0 | |
| Vitamins | B12, Choline chloride, Inositol | 0.5-15.0 | |
| Buffer | Sodium bicarbonate | 2.0-3.0 | g/L |
| Minerals | Calcium chloride, Magnesium | 25.0-150.0 | |
| | sulfate | | |
| Trace metals | Ferric nitrate, Ferrous, Zinc and | 0.001-0.6 | |
| | Cupric sulfate | | |
| Lipids & Fatty | Linoleic Acid | 0.03-0.3 | |
| acids | | | |
| Proteins | Collagens, Insulin, Transferrin | 0.1-3.0 | |
| Cell growth | | | |
| enhancers | | | |
| Growth factor | EGF | 0.1-10.0 | |
| Attachment factor | Fibronectin | 5.0-50.0 | |
| Stimulators | | | |
| Growth factor | TGF-β, VEGF | 0.1-10.0 | |
| Fibrous Protein | Elastin, collagen | 0.1-3.0% | |
| ECM stimulator | Ascorbate | 30-150 | |
| Adhesion protein | Hyaluronic acid | 1.0-20.0 | |
| Glucosamine | Heparin, chondroitin sulfate, | 0.1-10 | |
| glycan | aggrecan | | |
| Penetration | | | |
| Enhancers | | | |
| Alcohol | | 0-20.0 | |
| Others | | | |

| | |
|---|---|
| Water (USP) | Balance |

### EXAMPLE 4

This example provides a composition to promote hair growth on the scalp or to decrease the rate of hair loss on the scalp. The formulation is prepared as an oil, with preservatives, as illustrated below.

| COMPONENTS | INGREDIENT | Conc. Range (mg/L) | |
|---|---|---|---|
| Nutrients | | | |
| Sugar | D-Glucose | 2.0-6.0 | g/L |
| Amino acids | essential and non essential | 4.0-150.0 | |
| Pyruvate salt | Sodium Pyruvate | 50.0-60.0 | |
| Phosphate salts | Sodium phosphate | 50.0-80.0 | |
| Vitamins | selected | 0.5-15.0 | |
| Minerals | Calcium chloride, Magnesium | 25.0-150.0 | |
| | sulfate | | |
| Trace metals | selected | 0.001-0.6 | |
| Fatty acids | Linoleic Acid, Lipoic acid, | 0.03-0.3 | |
| | Oleic acid | | |
| Proteins | Insulin, Transferrin | 0.1-3.0 | |
| Cell growth | | | |
| enhancers | | | |
| Growth factor | selected | 0.1-10.0 | |
| Attachment factor | Hyaluronic acid | 1.0-20.0 | |
| ECM and | | | |
| Stimulators | | | |
| Growth factor | selected, VEGF | 0.1-10.0 | |
| ECM stimulator | Ascorbate | 30-150 | |
| Adhesion protein | Hyaluronic acid, Fibronectin | 5.0-50.0 | |
| Integrins | IL1, IL6 | 0.1-10 | |
| Penetration | | | |
| Enhancers | | | |
| Propylene glycol | | 0.1-4.0 | |
| Isopropanol | | 0-20.0 | |
| Tween 80 | | 0-5.0 | |
| Mineral oil | | 10-90% | |
| (liposomal | | | |
| emulsion) | | | |
| Others | | | |
| Herbal oils | | 10-90% | |
| Extracts | Xanthan gums | 0.1-1.0 | |

| | |
|---|---|
| Water (USP) | Balance |

### EXAMPLE 5

This example provides another illustration of a composition of the present invention for rejuvenating skin.

| | | | | Target | |
|---|---|---|---|---|---|
| | | Conc. Range | | conc., | |
| COMPONENTS | INGREDIENT | (mg/L) | | ug/L | |
| Nutrients | | | | | |
| Cell growth medium | DMEM/F12 | | | | |
| | (Invitrogen) | | | | |
| Fatty acid Soaps | Linoleic Acid | 0.03-0.3 | | 100 | |
| Proteins | Collagens | 0.1-3.0 | | 500 | |
| Cell growth | | | | | |
| enhancers | | | | | |
| Growth factor | EGF | 0.1-10.0 | | 5 | |
| ECM and | | | | | |
| Stimulators | | | | | |
| Growth factor | TGF-b | 0.01-1.0 | ug/L | 0.05 | |
| | VEGF | 0.1-10.0 | ug/L | 0.05 | |
| ECM stimulator | Ascorbate | 30-150 | | 150 | |
| | (Sodium | | | | |
| | L-ascorbate) | | | | |
| Adhesion protein | Hyaluronic acid | 1.0-20.0 | | 1000 | |
| Glucosamine glycan | Glucosamine | 0.1-1.0 | | 500 | |
| Penetration | | | | | |
| Enhancers | | | | | |
| Alcohols | Isopropanol | <1.0% | | 10 | ml/L |
| Glycols | Propylene glycol | <1.0% | | 10 | ml/L |
| Detergents | Tween 80 | <0.1% | | 1 | ml/L |
| Oils | Jojoba Oil | 1% | | 10 | ml/L |
| Others | | | | | |
| Bulking agents | Xanthan gums | 0.1-1.0 | | 1000 | |
| Preservatives | | | | | |
| Color | | | | | |

### EXAMPLE 6

This example provides another illustration of a composition of the present invention for rejuvenating skin.

| | | | | Target | |
|---|---|---|---|---|---|
| | | Conc. Range | | conc., | |
| COMPONENTS | INGREDIENT | (mg/L) | | ug/L | |
| Nutrients | | | | | |
| Cell growth medium | DMEM/F12 | | | | |
| | (Invitrogen) | | | | |
| Fatty acid Soaps | Oleic acid | 0.03-0.3 | | 100 | |
| | (canola oil soap) | | | | |
| Cell growth | | | | | |
| enhancers | | | | | |
| Growth factor | EGF | 0.1-10.0 | | 5 | |
| ECM and | | | | | |
| Stimulators | | | | | |
| Growth factor | TGF-β | 0.01-1.0 | ug/L | 0.05 | |
| | basic FGF-2 | 0.1-10.0 | ug/L | 0.05 | |
| ECM stimulator | Ascorbate | 30-150 | | 150 | |
| | (Sodium | | | | |
| | L-ascorbate) | | | | |
| Adhesion protein | Hyaluronic acid | 1.0-20.0 | | 1000 | |
| Penetration | | | | | |
| Enhancers | | | | | |
| Alcohols | Isopropanol | <1.0% | | 10 | ml/L |
| Glycols | Propylene glycol | <1.0% | | 10 | ml/L |
| Detergents | Tween 80 | <0.1% | | 1 | ml/L |
| Others | | | | | |
| Bulking agents | Xanthan gums | 0.1-1.0 | | 1000 | |
| Preservatives | | | | | |
| Color | | | | | |

### EXAMPLE 7

This example describes an in vitro study demonstrating transport into and through the skin of a composition of the invention using percutaneous absorption in human cadaver skin. The model uses human cadaver skin mounted in specially designed diffusion chambers, which allow the skin to be maintained at a temperature and humidity corresponding to in vivo conditions.

Human cadaver trunk skin, without obvious signs of skin disease, is obtained within 24 hours of death from a skin bank. The skin is dermatomed to approximately 0.25 mm, sealed in a water-impermeable plastic bag and stored at −70° C. until the day of the experiment. Prior to use the skin is thawed by placing the bag in a 37° C. water bath and rinsing it in tap water to remove adherent blood or other materials from the surface. Skin from a single donor is cut into multiple smaller sections large enough to fit on 2.0 cm² Franz diffusion cells. The dermal chamber is filled to capacity with a receptor solution of PBS, pH 7.4, and the epidermal chamber is left open. The Franz cells are then placed in a diffusion apparatus in which the dermal receptor solution is stirred magnetically at 600 rpm and its temperature is maintained at 37° C.

At the start of the study, the receptor solution is replaced with a fresh solution of PBS. Another solvent may be used in place of PBS if the composition is not soluble in water, to improve recovery. Each test composition is applied to triplicate sections (tape stripped skin, if exfoliation conditions need to be simulated) of donor skin at a target dose of 2-10 ml/cm². The donor chimney is covered with a water impermeable barrier (e.g. plastic wrap) to prevent evaporation. At 24 and 48 hours after dosing, the receptor solution is removed and replaced with fresh solution. The harvested receptor solution is analyzed for the presence and concentration of composition-components.

Total absorption of a component is measured by the total amount of material collected over 48 hours (in all receptor solutions) and a percent absorption is calculated from the amount applied. The rate of absorption of component material is calculated over the 48-hour period. The percent absorption and the rate of absorption of components are used to compare compositions for penetration characteristics. Detectable presence of composition components in the receptor solution demonstrates that the components of the compositions penetrate the skin barrier and will reach the inner layers of the skin, in vivo, when applied topically to the skin.

### EXAMPLE 8

This example describes an in vitro study for demonstrating sustained cell nourishment and an increase in the cell growth rate achieved with the use of the present invention. Concentrations of test composition permeating the skin are demonstrated, as determined from percutaneous absorption testing. DMEM/F12 cell culture medium is used as the control.

Human dermal fibroblast cells (obtained from a culture collection such as ATCC) are inoculated at 0.5 million cells in T-150 flasks containing 40 ml of DMEM/F 12 supplemented with 10% bovine calf serum and grown in a 5% CO₂ incubator at 37° C. Confluent T-flask culture (usually grown for 12 days) are trypsinized, and the cells counted and collected. 0.5 million human dermal fibroblast cells are pipetted into twenty four T150 flasks, each containing 40 ml of DMEM/F12 supplemented with 10% Bovine calf serum. The T-150 flasks are placed in a 5% CO₂ incubator at 37° C. Spent medium in the T-flasks is exchanged with 40 ml of fresh medium at day 4. At day 8, three T-flasks are removed from the incubator, cell morphology is noted, T-flasks are trysinized and the cells counted to get an average cell count. Twelve T-flasks are labeled as ‘Test Composition’ and twelve as ‘Control’. Spent medium is removed from all T-flasks. T-flasks are washed with 40 ml of PBS twice to remove traces of spent medium. 40 ml of the test composition is pipetted into the 12 T-flasks marked as ‘Test Composition’. 40 ml of DMEM/F12 is pipetted into the 12 T-flasks marked as ‘Control’. All flasks are placed in the incubator. At day 9, 10, 11 and 12, three T-flasks each labeled as ‘Test Article’ and ‘Control’ are removed, and flasks are observed for cell morphology. T-flasks are then trypsinized and cells counted.

Cell morphology for T-flasks containing the test composition is compared to control flasks and scored using a scale of 1-5. A statistically significant increase in maintenance of cell morphology over time for T-flasks containing the test composition versus the control demonstrates that the composition provides cell nutrition for improved cell maintenance, and thereby would be beneficial for skin cell care.

Cell growth rates for T-flasks containing the test composition is compared to control flasks. A 5-25% increase in cell growth rate for T-flasks containing test composition versus the control demonstrates that the composition provides enhancement of cell metabolism, and is thereby is beneficial for skin cell care.

Using the conventional definition with alpha=0.05, a result is statistically significant when the result would occur less than 10% of the time if the populations were really identical.

### EXAMPLE 9

This example describes an in vitro study demonstrating stimulation of extracellular matrix protein production and deposition of extracellular matrix proteins in fibroblasts according to the present invention. Concentrations of test composition permeating the skin, as determined in the percutaneous absorption model, are determined in this study. Dulbecco's Modified Eagle's Medium/Ham's F12 50/50 mix (DMEM/F12) cell culture medium is used as the control.

Human dermal fibroblast cells (obtained from a culture collection such as ATCC) are inoculated at 0.5 million cells in T-150 flasks containing 40 ml of DMEM/F12 supplemented with 10% bovine calf serum and grown in a 5% CO₂ incubator at 37° C. Confluent T-flask culture (usually grown for 12 days) are trypsinized, cells are counted and collected. 0.5 million human dermal fibroblast cells are pipetted into thirty T150 flasks, each containing 40 ml of DMEM/F12 supplemented with 10% bovine calf serum. The T-150 flasks are placed in a 5% CO₂ incubator at 37° C. Spent medium in the T-flasks is exchanged with 40 ml of fresh medium at day 4 and day 8. At day 12, all T-flasks are observed for confluency and only flasks that are 100% confluent are kept in the study. Three T-flasks are extracted with trypsin/EDTA/SDS mix to remove cells and extracellular matrix from the flasks. The extract is analyzed for quantitative measurement of collagen and GAG's to get an average baseline for ECM production. Twelve T-flasks are labeled with ‘Test Composition’ and twelve as ‘Control’. 40 ml of test composition is pipetted into the 12 T-flasks marked as ‘Test Composition’. 40 ml of DMEM/F12 is pipetted into the 12 T-flasks marked as ‘Control’. All flasks are placed in the incubator. At day 9, 10, 11 and 12, three T-flasks each labeled as ‘Test Composition’ and ‘Control’ are removed, flasks are extracted and analyzed for ECM production.

Total extracellular matrix (ECM) production and ECM production rates for T-flasks containing test composition is compared to Control flasks. A 5-50% increase in total ECM and ECM production rate for test composition T-flasks versus Control flasks demonstrates that the composition stimulates ECM production.

### EXAMPLE 10

This example describes an in vivo study demonstrating that the compositions of the invention moisturize skin in normal subjects with dry skin. This provides a measure of skin texture. Four different formulations of the invention are used as active compositions (Test Compositions A-D). Phosphate buffer saline is used as the negative control (Control).

Fifteen Caucasian female subjects, 30-50 years of age, are entered into the study and undergo a 7 day “wash out” phase in which the lower legs are washed with Ivory® soap (Proctor & Gamble, Cincinnati, Ohio) twice a day and refrained from using any moisturization products on their lower legs.

After the seven-day “wash-out” period the subjects start the treatment with a 30 minute equilibration phase in an environment of 70+/−3° F. and a relative humidity of less than 30%. The lower legs of all subjects are evaluated for dryness to qualify for the study. Subjects with no dryness are eliminated from the study group. The subjects enrolled in the study have six 4×6 cm squares marked on the outer aspect of the lower leg that had the driest skin. The test compositions are applied to the sites using a standard rotation to eliminate positional bias.

At time zero, approximately 20 μl of the test composition is applied to the appropriate test site with a latex finger cot. The sixth test site is kept as the untreated site. The subjects remain in the testing facility for the duration of the study in a quiet fashion without drinking excessive water, smoking or eating. Skin moisturization readings are taken in duplicate using a NOVA DPM meter 9003 at each test site approximately one, two, three and four hours after application of the test composition. D-SQUAME® discs (Cuderm Corp., Dallas, Tex.) are taken at baseline on the untreated site and at four-hour evaluation on all test sites to measure the amount of stratum corneum scaling. The D-SQUAME® discs are then imaged to determine skin moisture readings.

The desquamation index is known in the art as an indicator of dry skin flakiness. The greater the number, size, and thickness of dry skin flakes, the higher the desquamation index. A 15% or greater increase in skin moisturization reading and a 10% or greater decrease in the Desquamation Index for test compositions versus the control demonstrates that the compositions moisturize skin and thereby improve skin texture and provide a more youthful skin appearance. Various computer programs and instrumentation is available in the art to conveniently determine the desquamation index. The programs preferably apply a mask to the image to define a measurement area of 200 mm². A lookup table is applied to the image, which substitutes a new set of numeric values for the default gray scale so that ranges of gray levels are represented by single values. Each pixel is assigned to one of five arbitrary thickness levels of the corneocyte clusters. Transformations can be used to calculate the number of pixels in each thickness group as a percent value as well as the total area occupied with cells. The percentage area occupied by corneocytes is also determined. These two functions are integrated to yield the desquamation index according to the following formula:

$$D . I . = \frac{2 A + {\sum }_{n = 1}^{5} Tn * ( n - 1 )}{6}$$

where D.I. is the desquamation index, A is the percent area covered by comeocytes, Tn is the percentage of comeocytes in relations to thickness, and n is the thickness level (1-5).

### EXAMPLE 11

This example describes an in vivo study designed to evaluate the compositions for effectiveness in rejuvenating the skin, by reducing the occurrence of facial lines in a double-blind vehicle controlled study. A formulation of the invention is used as an active composition (Test Composition). Phosphate buffer saline is used as the vehicle control (Control).

Thirty females ages 35-75, selected for the study based on good general health are entered into the in the study and undergo a 7 day “wash out” period in which they refrain from using all facial moisturizing products (i.e. soaps, creams, lotions, gels). The subjects are given a non-moisturizing glycerin soap (Neutrogena Corp., Los Angeles, Calif.) to use for their daily cleansing of the face throughout the “wash-out and treatment” phases. The subjects are allowed to use their regular make-up during the wash-out period and for the duration of the study.

On day 1 after the “wash-out” period, 35 mm color photographs are taken (with a 70-300 mm macro lens) of each side of the face followed by silicone replicas from the crow's feet area. The procedure for taking replicas is as follows: two or three drops of catalyzer is added to the SILFLO® resin (Silflo, Flexico Developments Ltd., Potters Bar, England) and is rapidly mixed. The paste is then immediately applied to the skin surface. After two or three minutes, the replica is gently removed from the skin and moisture readings recorded from a NOVA DPM meter 9003, or equivalent instrument that measures moisture content of skin by electric conductance.

Additional replicas are taken from the cheek area (duplicate reading from each cheek) to determine the level of the skin surface moisture. Also in the cheek area D-SQUAME® tape is used to take specimens of the outer surface of the stratum corneum in order to measure the effect of the test composition on reducing skin roughness. Each subject is given product to use at home. The assignment of Test Composition and Control is alternated from subject to subject. Each subject is given a diary to take home to record the time of each application as well as to report any sensory or visual irritation. The subjects return after four and eight week intervals to have their diaries checked and to receive more Test Composition or Control as necessary. The subjects return to the testing facility after a total of 12 weeks of treatment for final silicone replicas, photographs, NOVA readings, and to have D-SQUAME® tape applied to the skin.

The D-SQUAME® tape and silicone replicas are analyzed by electronic imaging and image analysis to determine the desquamation index. A statistically significant increase in the skin moisturization reading and a decrease in the Desquamation Index for test composition subjects versus control subjects demonstrates that the compositions increase skin moisturization and thereby improve skin texture and enhance a youthful skin appearance. A reduction in the number of fine lines assessed by image analysis of the silicone replicas of subjects using test composition versus Control demonstrates that the compositions reduce fine lines and wrinkles, and thereby strengthen the skin structure.

### EXAMPLE 12

This example describes an in vivo study designed to illustrate the present compositions' effectiveness in wound healing in human subjects in a double-blind vehicle controlled study. A composition formulated for wound healing application is used as a the test composition.

Wounds are created in the forearms of eight human volunteers using a keratome with a setting of approximately 0.3 mm. A measurement of the width of the wound is taken (in mm). Treatment is begun immediately after wounding and twice a day for the next four days. Photographs of the wound and clinical observations are conducted daily.

On the second and fourth days after wounding, a measurement of the width of the wound is taken along with a 2 mm biopsy. The biopsy is processed according to standard procedures and the tissue sections are stained with H&E. A dermal pathologist evaluates the histological sections for vascularization, inflammation and epithelialization. Wound healing capabilities of the test composition is demonstrated by any of the following criteria: a 10-30% reduction in wound width measurement, an increase in vascularization of the wound, a reduction in inflammation, or an increase in epithelialization using the composition versus the control.

### EXAMPLE 13

This example describes an in vivo study designed to demonstrate the present invention's effectiveness in increasing hair growth on the scalp of human subjects, or in decreasing the rate of hair loss on the scalp. The study is a double-blind vehicle controlled study. A composition formulated for hair growth application is used as an active composition.

Approximately 30 men, ages 18 to 40 with alopecia androgenetica as evidenced by frontal/parietal hair thinning, as defined by the Hamilton Scale as Type III or IV, are enrolled in the study.

A representative site on the thinning frontal/parietal scalp is chosen as the treatment site. The site is marked using permanent ink at the four corners. The hair is carefully clipped from the test site, counted and weighed. After six weeks the process is repeated and the treatment is begun.

The subjects return to the test facility every six weeks for six months to have the hair in the treatment site clipped and weighed. A 5-25% increase in weight of hair clippings or hair count among test composition subjects versus control subjects demonstrates that the compositions increase hair growth on the scalp, and that the compositions will decrease the rate of hair loss on the scalp.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are set forth within the following claims.

1. A composition for skin repair comprising:
an amount of at least one cell growth enhancer in an amount effective to increase the growth rate of skin cells; and
nutrients in an amount effective to support log phase growth of skin cells; and
at least one extra-cellular matrix protein; and
at least one stimulator of extra-cellular matrix protein production in an amount effective to increase the production of extra-cellular matrix proteins in the skin; and
at least one penetration enhancer in an amount effective to allow the penetration of the cell growth enhancers, nutrients, extracellular matrix proteins, and stimulators of extracellular matrix protein production through mammalian skin in amounts effective to promote skin repair;
in a biologically acceptable carrier.

2. The composition of claim 1 wherein:
the at least one growth enhancer is selected from the group consisting of: epidermal growth factor (EGF), fibroblast growth factor (FGF), insulin-like growth factor (IGF), granulocyte colony stimulating factor (GCSF), granulocyte macrophage colony stimulating factor (GMCSF), platelet derived growth factor (PDGF), keratinocyte growth factor (KGF), tissue growth factor-α (TGF-α), vascular endothelial growth factor (VEGF), erythropoictin, hematopoietin, growth hormone, prostaglandin, cytokines, regulatory factors, angiogenic factors, hyaluronic acid, and fibronectin; and
the nutrients effective to support log phase growth of skin cells are selected from the group consisting of: monosaccharides, dissaccharides, carbohydrates, essential and non essential amino acids, salts, vitamins, minerals, trace metals, nucleosides, purines, pyrimidines, glutathione, peptides, peptones, lipoproteins, and fatty acids; and
the at least one extra-cellular matrix protein is selected from the group consisting of: fibrous proteins, adhesion proteins, glucosamineglycans, proteoglycans, and integrins; and
the at least one stimulator of extra-cellular matrix protein production is selected from the group consisting of: tissue growth factor-β, and adhesion proteins; and
the at least one penetration enhancer is selected from the group consisting of: mineral oil, fatty alcohols, detergents, alcohols, glycols, lipoic acid, a transdermal delivery vehicle, or a transdermal delivery device.

3. The composition of claim 1 wherein
the nutrients effective to support log phase growth of skin cells are selected from the group consisting of: monosaccharides, carbohydrates, essential and non essential amino acids, salts, vitamins, minerals, trace metals, nucleosides, purines, pyrimidines, glutathione, peptides, peptones, lipoproteins, and fatty acids.

4. The composition of claim 1 wherein the at least one extra-cellular matrix protein is selected from the group consisting of: fibrous proteins, adhesion proteins, glucosamineglycans, proteoglycans, and integrins.

5. The composition of claim 1 wherein the at least one stimulator of extra-cellular matrix protein production is selected from the group consisting of: tissue growth factor-β, and adhesion proteins.

6. The composition of claim 1 wherein the at least one penetration enhancer is selected from the group consisting of: mineral oil, fatty alcohols, detergents, alcohols, glycols, lipoic acid, a transdermal delivery vehicle, or a transdermal delivery device.

7. A composition for skin repair comprising:
at least one cell growth enhancer selected from the group consisting of: epidermal growth factor, fibroblast growth factor, tissue growth factor-α, hyaluronic acid, fibronectin, hepatopoietin, erythropoietin, growth hormone, prostaglandin, VEGF, fibronectin, vitronectin, laminin, and tenasin; and
at least one nutrient selected from the group consisting of: D-glucose, amino acids, sodium chloride, potassium chloride, sodium pyruvate, vitamin B12, choline chloride, inositol, calcium chloride, magnesium sulfate, ferric nitrate, ferrous sulfate, zinc sulfate, cupric sulfate, hypoxanthine, linoleic acid, lipoic acid, inositol, oleic acid, collagen, insulin, and transferrin; and
at least one extracellular matrix protein selected from the group consisting of: collagen, elastin, and transferrin; and
ascorbate; and
at least one penetration enhancer selected from the group consisting of: propylene glycol, a fatty alcohol, polyoxyethylene sorbitan monooleate (Tween 80™), butylene glycol, mineral oil, and a TAT protein sequence attached to a component protein.

8. A method for repairing mammalian skin comprising:
contacting the skin with a composition of claim 1:
allowing the composition to remain in contact with the skin for a period of time sufficient for the cell growth enhancers, nutrients, extracellular matrix proteins, and stimulators of extracellular matrix to permeate mammalian skin in amounts effective to repair the skin;
thereby repairing the mammalian skin.

9. The method of claim 8 wherein the repair is the rejuvenation of the skin.

10. The method of claim 8 wherein the repair is the healing of a wound.

11. The method of claim 9 wherein the rejuvenation of the skin comprises a reduction in fine lines and wrinkles of the treated skin by 10% or more.

12. The method of claim 8 wherein the mammalian skin comprises hair follicles.

13. The method of claim 12 wherein growth of hair from the hair follicles increases by 10% or more.

14. The method of claim 10 wherein the wound is a sunburn or a topical abrasion.

15. The method of claim 8 wherein the composition is applied as a coating on a medical or surgical device selected from the group consisting of: sutures, implants, homeostatic plugs, dressings, gauze and pads.

16. A method for repairing mammalian skin
contacting the skin with a composition of claim 7;
allowing the composition to remain in contact with the skin for a period of time sufficient for the cell growth enhancers, nutrients, extracellular matrix proteins, and stimulators of extracellular matrix to permeate mammalian skin in amounts effective to repair the skin;
thereby repairing the mammalian skin.

17. A method for increasing hair growth on the scalp comprising:
contacting the skin of the scalp with a composition of claim 7;
allowing the composition to remain in contact with the skin for a period of time sufficient for the cell growth enhancers, nutrients, extracellular matrix proteins, and stimulators of extracellular matrix to permeate mammalian skin in amounts effective to increase hair growth;
thereby increasing the growth of hair on the scalp.
